(19) United States
(12) Patent Application Publication
(10) Pub. No.: US 2017/0215757 A1
(43) Pub. Date: Aug. 3, 2017

(54) PHYSIOLOGICAL SIGNAL DETECTION AND ANALYSIS SYSTEMS AND DEVICES

(71) Applicant: NEUROVERSE INC., San Diego, CA (US)

(72) Inventors: Ricardo GIL DA COSTA, San Diego, CA (US); Raynard FUNG, San Diego, CA (US)

(21) Appl. No.: 15/314,916

(22) PCT Filed: May 29, 2015
(86) PCT No.: PCT/US15/33383

§ 371 Date: Nov. 29, 2016

Related U.S. Application Data
(60) Provisional application No. 62/004,667, filed on May 29, 2014.
(60) Provisional application No. 62/019,892, filed on Jul. 2, 2014.

(51) Int. Cl. A61B 5/0476; A61B 3/113; A61B 5/16; A61B 5/00; A61B 5/11; A61B 5/0488; A61B 3/00

(52) U.S. Cl. CPC A61B 5/0476; A61B 5/0488; A61B 3/113; A61B 3/0041; A61B 5/6803; A61B 5/1102; A61B 5/0015; A61B 5/16; A61B 2560/0214; A61B 2560/0431; A61B 5/6833

(57) ABSTRACT

In one aspect, a sensory, motor and/or cognitive analysis device includes a casing unit structured to include a contact side conformable to a forehead region of a user; a data acquisition unit structured to include one or more sensors to detect electrophysiological signals of the user when the user makes contact with the device; a data processing unit encased within the casing unit and in communication with the data acquisition unit to amplify and digitize the detected electrophysiological signals as data, to process the data, to store the data, and to transmit the data to a remote computer system; and a power supply unit encased within the casing unit to provide electrical power, in which the device is operable to acquire physiological and/or behavioral signal data from the user used to determine a quantitative and/or qualitative information set associated with a cognitive or sensory assessment.

### CROSS-REFERENCE TO RELATED APPLICATIONS

This patent application document claims the benefit of priority of U.S. Provisional Application No. 62/004,667, entitled “PHYSIOLOGICAL SIGNAL DETECTION AND ANALYSIS SYSTEMS AND DEVICES,” filed on May 29, 2014, and U.S. Provisional Application No. 62/019,892, entitled “PHYSIOLOGICAL SIGNAL DETECTION AND ANALYSIS SYSTEMS AND DEVICES,” filed on Jul. 2, 2014. The entire content of the above mentioned patent applications are incorporated by reference as part of the disclosure of this patent document in their entirety for all purposes.

### BACKGROUND

Establishing reliable correlations between one's physiological signals and the associated cognitive/psychological states may enable valuable and desired applications for various uses, such as in clinical and consumer electronics domains, amongst others. Such correlations, extensively explored in fundamental sciences, have been the focus of various translational attempts into specialized applications such as assessment of cognitive impairment and enabling the physically impaired to communicate.

Several factors may be used to determine sensory and/or cognitive information about a subject. For example, such factors may include the type of physiological signals and/or behavioral response to detect and measure, the type of stimuli to evoke the subject's response, duration of the stimuli, inter-stimuli interval, number of repetitions of each presentation of stimuli, the levels of the stimuli (e.g., sound, brightness or contrast levels, etc.), markers associated with the onset of presentation of each stimuli, etc., as well as the recording sensors and systems. Also, the physiological parameter(s) of use (e.g., voltage, power, frequency, etc.), the related time window for analysis, and the analysis structure can affect the brain signal recordings and correlated cognitive assessment. Deviations or mistakes from one or multiple of these parameters can make the difference between a useful or an artifact driven, useless device, system, application, and/or method.

One exemplary type of physiological signal that can be used in sensory and/or cognitive analysis is electroencephalography (EEG). EEG is the recording of electrical activity exhibited by the brain using electrodes positioned on a subject's head, forming a spectral content of neural signal oscillations that comprise an EEG data set. For example, the electrical activity of the brain that is detected by EEG techniques can include voltage fluctuations that may result from ionic current flows within the neurons of the brain. In some contexts, EEG refers to the recording of the brain's spontaneous electrical activity over specific periods of time. EEG can be used in clinical diagnostic applications including epilepsy, coma, encephalopathies, brain death, and other diseases and defects, as well as in studies of sleep and sleep disorders. In some instances, EEG has been used for the diagnosis of tumors, stroke and other focal brain disorders.

One example of an EEG technique includes recording of event-related potentials (ERPs), which refer to EEG recorded brain responses that are correlated with a given event (e.g., simple stimulation and complex processes). For example, an ERP includes an electrical brain response—a brain wave—related to sensory, motor, and/or cognitive processing. ERPs can be associated with brain measures of perception (e.g., visual, auditory, etc.) and cognition (e.g., attention, language, decision making, etc.). A typical ERP waveform includes a temporal evolution of positive and negative voltage deflections, termed components. For example, typical components are classified using a letter (N/P: negative/positive) and a number (indicating the latency, in milliseconds from the onset of stimulus event), for which this component arises.

### SUMMARY

According to aspects of the disclosure, a sensory, motor and/or cognitive analysis device is presented. The device includes a casing unit configured to include a contact side conformable to a forehead region of a user. The device also includes a data acquisition unit configured to include one or more sensors to detect electrophysiological signals of the user when the user makes contact with the device. The device also includes a data processing unit defined within the casing unit and in communication with the data acquisition unit. The data processing unit is configured to include a signal processing circuit to amplify and digitize the detected electrophysiological signals as data, a processor to process the data, a memory to store the data, and a transmitter to transmit the data to a remote computer system. The device also includes a power supply unit defined within the casing unit and electrically coupled to the data processing unit to provide electrical power. The device may be operable to acquire physiological and/or behavioral signal data from the user. The signal data may be used to determine a quantitative and/or qualitative information set associated with a cognitive and/or sensory assessment.

According to aspects of the disclosure, a sensory, motor and/or cognitive analysis device is presented. The device includes a housing configured to include a section receiving contact from a forehead of a user. The device also includes a data acquisition unit defined in the section of the housing and configured to include one or more sensors to detect electrophysiological signals of the user when the user makes the contact with the device. The device also includes a data processing unit located in the housing and in electrical communication with the data acquisition unit. The data processing unit is configured to include a signal processing circuit to amplify the detected electrophysiological signals as data and a transmitter to transmit the data to a remote computer system. The device may be operable to acquire brain signal data from the user, and a quantitative and/or qualitative information set associated with a cognitive and/or sensory assessment may be based on the signal data.

Additional features and advantages of the disclosure will be described below. It should be appreciated by those skilled in the art that this disclosure may be readily used as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 illustrates an example diagram for various aspects of neural testing and various possible assessments of mental and neural processes for enabling the creation of individual and/or group user quantitative and/or qualitative neurocognitive profiles according to aspects of the present disclosure.

FIGS. 2A and 2B illustrate block diagrams of exemplary devices according to aspects of the present disclosure.

FIG. 3 illustrates a block diagram of an exemplary data processing unit according to aspects of the present disclosure.

FIGS. 4A and 4B illustrate three-dimensional schematic diagrams of an exemplary portable device with a detachable data acquisition unit from an encased data processing unit according to aspects of the present disclosure.

FIG. 5 illustrates three-dimensional schematic diagrams of the exemplary portable device of FIGS. 4A and 4B showing a retractable cover of the casing structure of the data processing unit to receive an electrical connection tether to the detachable data acquisition unit according to aspects of the present disclosure.

FIG. 6 illustrates three-dimensional schematic diagrams of the exemplary portable device of FIGS. 4A and 4B showing a retractable cover of the casing structure of the data processing unit to receive a connector for power and/or data transfer according to aspects of the present disclosure.

FIG. 7 illustrates an exploded three-dimensional schematic diagram of an exemplary data acquisition unit of the exemplary portable device of FIGS. 4A and 4B according to aspects of the present disclosure.

FIG. 8 illustrates an exploded three-dimensional schematic diagram of an exemplary data processing unit and data acquisition unit of the exemplary portable device of FIGS. 4A and 4B according to aspects of the present disclosure.

FIGS. 9A-9H illustrate display screens of an exemplary user interface of a software application according to aspects of the present disclosure.

### DETAILED DESCRIPTION

Exemplary embodiments of the present invention will be described with reference to the accompanying drawings. The accompanying drawings have been made only for a better understanding of the embodiments of the present invention, and are not intended to limit the scope and spirit of the invention.

Due to the specialized structure and level of precision associated with operation of conventional physiological data acquisition and analysis systems (e.g., for stimulation, acquisition, recording and/or analysis of these signals), new technological systems and approaches may be created to improve one or more of the specified tasks. The disclosed technology includes techniques, systems, and devices with integrative hardware, firmware, and/or software for stimulation, acquisition, recording and/or analysis of physiological signals, factors, and/or parameters. In one example, an integrative system is disclosed that includes both hardware (sensors and devices) and software (such as user interface, user control, computer applications, and analysis) for acquiring, processing, and/or transmitting data to provide relevant and actionable cognitive and/or behavioral assessment of a user or group of users. This system—an integration of hardware and software—is a technological embodiment of broader methodologies (detailed below) and can be used in a variety of applications, e.g., including but not limited to, clinical, educational, safety, gaming, music, sports, and industrial uses, for both lay or professional/specialized users.

Devices, systems, and methods are disclosed for physiological and/or behavioral signals acquisition and analysis for determining sensory, motor, emotional and/or cognitive information of a subject, using an integration of hardware and/or software. The disclosed devices, systems, and methods use an array of electrode sensors positioned in a specialized configuration about the subject's forehead in tandem with a powered circuit board that improves the physiological signal detection and processing to produce various user assessments such as psychological states and/or behavioral preferences, among other various other types of cognitive and/or sensory processes evaluation, as well as brain-computer interface operations. Devices of the disclosed technology have been designed and may be configured in both portable and non-portable design forms that provide easy and user-friendly operation and comfort-of-use. Systems of the disclosed technology have been designed and implemented using firmware and/or software to allow a user or operator to easily interface with the functional units of the system, allowing them to perform multiple operations, including but not limited to acquire, transmit, assess, and access interpretation of the physiological and/or behavioral data. The disclosed technology may be used in a variety of health, education, entertainment, safety, and marketing applications.

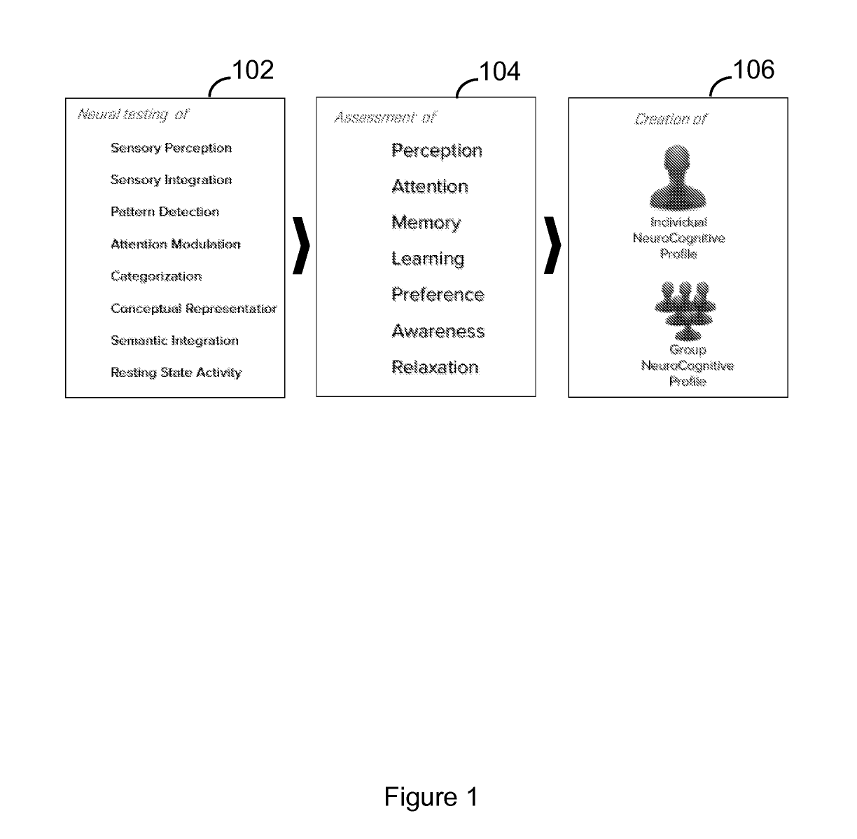

In one aspect, a device, such as a sensory and cognitive analysis device, includes an casing unit configured to include a contact side conformable to a forehead region of a user. The device may also include a data acquisition unit configured to include sensors to detect electrophysiological signals of a user, such that the data acquisition unit is in contact with the forehead region during the detection when the user uses the device. Furthermore, the device may include a data processing unit defined within the casing unit and in communication with the data acquisition unit. In one example, the data processing unit may be configured to include a signal processing circuit to amplify and digitize the detected electrophysiological signals as data, a processor to process the data, a memory to store the data, and/or a transmitter to transmit the data to a remote computer system. The device may also include a power supply unit defined within the casing unit and electrically coupled to the data processing unit to provide electrical power. Additionally, the device is operable to acquire physiological signal data from the user used to determine a quantitative information set associated with a cognitive, motor, and/or sensory function. FIG. 1 shows an example diagram depicting various aspects of neural testing and various possible assessments of mental and neural processes that can be performed using the disclosed technology to produce individual and/or group user quantitative and/or qualitative neurocognitive profiles. As shown in FIG. 1, using the described technology enables the physiological testing of aspects such as, but not limited to, sensory integration and categorization leading to assessments of user processes such as, but not limited to, learning and memory, resulting in the creation of individual and/or group neurocognitive profiles.

In another configuration, the device, such as a sensory, motor and cognitive analysis device, includes a housing configured to include a section that may receive contact from a forehead of a user. The device may also include a data acquisition unit defined in the section of the housing and configured to include sensors to detect electrophysiological signals of a user when the user makes the contact with the device. The device may also include a data processing unit located in the housing and in electrical communication with the data acquisition unit Furthermore, the data processing unit may be configured to include a signal processing circuit to amplify the detected electrophysiological signals as data and a transmitter to transmit the data to a remote computer system. Additionally, the device may be operable to acquire physiological signal data from the user. Finally, a quantitative information set associated with a cognitive, motor, and/or sensory function may be based on the acquired data.

Physiological signal data that the disclosed devices and systems can acquire to determine the quantitative information set may include electrophysiological data (e.g., EEG and EMG) and behavioral response data (e.g., such as eye movement, facial movements, and other behaviors) indicative of sensory, motor, and/or cognitive function. For example, in the case of EEG, the acquired EEG data can be used to assist in the determination of a mental state (e.g. focus, stress, relaxation, etc.), cognitive ability (attention, memory, decision making, etc.), as well as for clinical diagnostic applications to provide information on a neurological disorder or pathology (e.g., such as epilepsy, coma, encephalopathies, brain death, and other diseases and defects), as well as on sleep and sleep disorders, or for the diagnosis of tumors, stroke and other focal brain disorders.

In some embodiments, the device can be configured as an independent, wirelessly communicative, portable device capable of acquiring, processing, and/or transmitting data from a user wearing the device in a variety of unrestrictive environments (e.g., such as the user's home, place of activity such as work and play, transportation, etc.), including when the user is stationary and when the user is moving. The portable device can be configured to wirelessly transmit data (e.g., including raw or processed data) to another computer system or communication network (referred to as “the cloud”) that includes one or more remote computational processing and storing devices (e.g., servers in the cloud). In some embodiments, the device may be portable and the data acquisition unit may be a detachable unit including disposable electrode sensor assembly, e.g., such as an adhesive patch with embedded electronic sensors, that attachably/detachably couples to the data processing unit. In some embodiments, the data acquisition unit may include a non-detachable and moveable assembly protruding from an outer casing of the housing of the data processing unit.

In some embodiments, the device may be configured as a wired, stationary device in which the data processing unit is communicatively wired to a computer to process the acquired physiological signal and/or behavioral response data, and/or to transfer such data to another computer system or communication network (e.g., the cloud). In some embodiments of the wired device, the data acquisition unit is a detachable unit including disposable electrode sensor assembly, e.g., such as an adhesive patch with embedded electronic sensors, that attachably/detachably couples to the data processing unit of the stationary device. In some embodiments of the wired device, the data acquisition unit includes a non-detachable and moveable assembly protruding from an outer casing of the housing of the data processing unit of the stationary device. For example, the non-detachable, moveable assembly may include electrodes housed in a moveable containment structure that protrudes from and is moveable with respect to the casing of the data processing unit.

The disclosed devices and systems may provide an evaluation of a variety of sensory and cognitive aspects of an individual subject and/or an evaluation of sensory and cognitive aspects of multiple subjects comprising a group, using an integration of the disclosed hardware and software technology. Examples of the cognitive aspects and physiological/health information that can be provided by such evaluations include, but are not limited to, evaluation of cognitive state, knowledge, learning mechanisms, behavioral preferences, vulnerability, and/or symptoms of neurological and neuropsychiatric pathologies.

In some embodiments, for example, the disclosed devices and systems include sensors to acquire frontal EEG recordings by configuring electrodes about the device to contact a user's forehead for physiological signal monitoring in such a manner that does not overlap past the user's hairline. In some implementations, for example, the sensors are configured to be a small size and coupled to an outer casing of the body of the device, e.g., via a moveable sensor-housing structure to adapt to varying curvatures on any user's forehead. In another conifguration the sensors are configured to be a small size and contained in a disposable unit that may be detachably coupled to the body of the device (e.g., thereby permitting one-time or limited-use of the device to one or a limited number of users). The sensors may be formed with a variety of different materials (e.g., conductive materials including but not limited to gold, silver, or silver chloride), which can be tailored for specific applications. The materials may be selected such that the device may have a reduced evasiveness when applied by the user.

The disclosed devices, systems, and methods may be implemented to provide a cognitive performance profile, a sensory performance profile, and/or a cognitive and sensory performance profile indicative of a subject's cognitive and/or sensory ability at the time of the assessment, using an integration of the disclosed technological hardware and/or software. For example, using the disclosed software, the type of cognitive and/or sensory profile may be selected by the user (e.g., such as the subject or a system operator) to provide a set of information 104 including a quantitative level of cognitive and/or sensory performance, e.g., including, but not limited to perception, attention, memory, learning, preference, state of awareness, and/or state of relaxation. The levels may be based on neural testing 102 of sensory perception, sensory integration, pattern detection, attention modulation, categorization, conceptual representation, semantic integration, and/or resting state activity. The basis of testing is not so limited however. The neural testing may also be based on confabulation, as well as others. Such quantitative data may include numerical values indicative of the cognitive and/or sensory performance, as well as brief textual description of the cognitive and/or sensory performance, and/or graphical representations of the cognitive and/or sensory performance In some implementations, the disclosed devices and systems can be implemented to provide the cognitive and/or sensory profile 106 using only physiological data acquired from the subject, e.g., with no overt behavioral response elicited from the subject. In some implementations, the disclosed devices and systems can be implemented to provide the cognitive and/or sensory profile including previously acquired physiological data from the subject, or other subjects (e.g., group data). The disclosed devices and systems can thereby, for example, be implemented to provide a cognitive and/or sensory profile about a group, 106.

Examples of methods to determine an information set of sensory and/or cognitive performance are described in PCT Publication WO 2014/052938 A2, entitled “SYSTEMS AND METHODS FOR SENSORY AND COGNITIVE PROFILING”, incorporated by reference in its entirety. In one example, the device may be used to generate the information set associated with the cognitive and/or sensory profile using the acquired physiological data and/or behavioral data. Specifically, the methods to determine an information set of sensory and/or cognitive performance may be implemented on the disclosed devices and systems The device may include the data processing unit. Furthermore, the device may include remote computer systems (e.g., including remote computers, user computer/communication devices including smartphones, tablets, and/or wearable devices, and cloud computer systems). The method may also include using physiological and/or behavioral data acquired from the subject previously using the disclosed devices and systems, or from one or more groups of individuals using the disclosed devices and systems that include or do not include the subject. The method may include a process to identify a time interval associated with the physiological signals (and/or behavioral signal data) based upon the presented stimuli and the selected profile category. For example, a time interval may include contiguous, discontinuous, continuous, discrete, or single time points. The method may include a process to group the data (e.g., physiological and/or behavioral) corresponding to the time interval into one or more grouped data sets. As another example, the process may include grouping the physiological data (and/or behavioral data) based on a pre-assigned category of the individual stimulus and/or an associative relationship of consecutive stimuli. The method may include a process to provide a statistical measure of a relationship across or within the grouped data sets to generate the one or more quantitative values for the selected profile category. In some implementations, for example, the method may include a process to enhance the signal of the physiological (and/or behavioral data) in the grouped data sets.

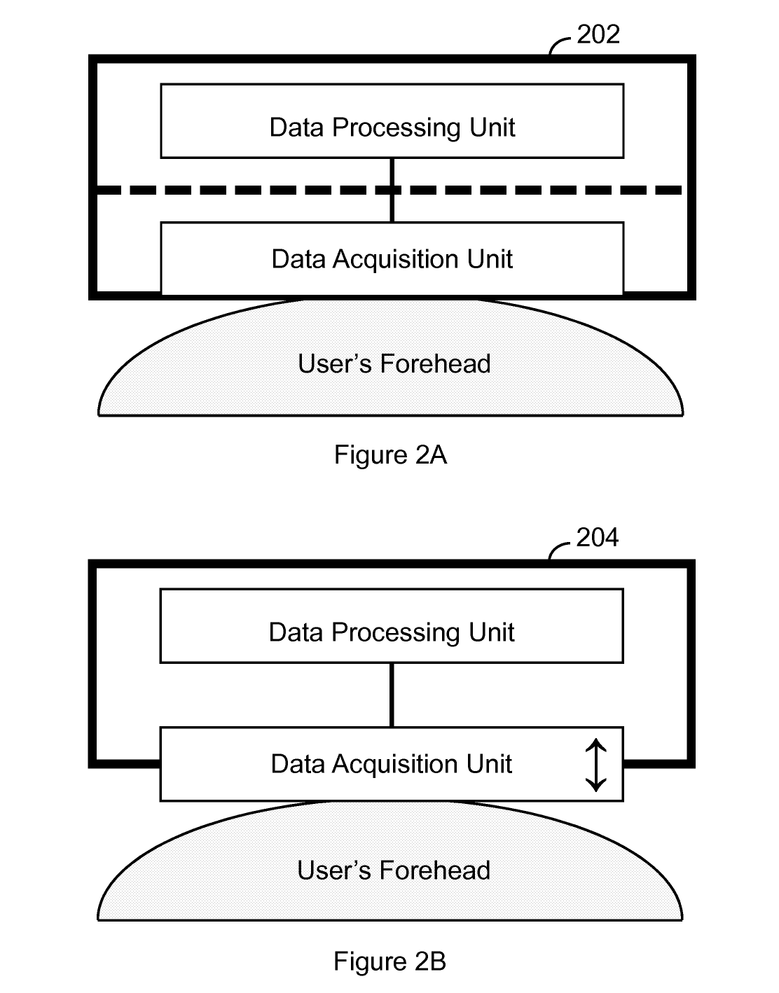

FIGS. 2A and 2B show block diagrams of exemplary sensory and cognitive analysis devices according to certain aspects of the disclosed technology. The exemplary device of FIG. 2A includes a data processing unit communicatively coupled to a data acquisition unit configured to contact a user's forehead. The data processing unit is encased in a casing structure 202 or housing. In some embodiments, the data acquisition unit is at least partially encased in the casing, whereas in other embodiments the data acquisition unit is attached to the casing 204 (e.g., which can be disposable and detachably attached).

In one exemplary embodiment of the sensory and cognitive analysis device shown in FIG. 2A, the device includes a casing unit 202 configured to include a contact side conformable to a forehead region of a user; a data acquisition unit configured to include one or more sensors to detect electrophysiological signals of a user when the user makes contact with the device; a data processing unit encased within the casing unit and in communication with the data acquisition unit, the data processing unit configured to include a signal processing circuit (e.g., including an amplifier and an analog-to-digital unit) to amplify and digitize the detected electrophysiological signals as data, a processor to process the data, a memory to store the data, and a transmitter to transmit to the data to a remote computer system; and a power supply unit encased within the casing unit 202 and electrically coupled to the data processing unit to provide electrical power, in which the device may acquire physiological signal data from the user, and wherein a quantitative information set associated with a cognitive, motor and/or sensory function may be based on the signal data.

Implementations of the exemplary device can may include one or more of the following exemplary features. For example, the detected electrophysiological signals can may be EEG signals measured from the brain of the user. In such examples, the data processing unit can be configured to determine the quantitative information set based on the measured EEG signals associated with an ERP. For example, the power supply unit can include a rechargeable battery, in which the device can further include an interface assembly configured to include an electronic interface electrically coupled to the battery and a retractable cover attached to the casing unit to expose and cover the electronic interface. In such examples, the electronic interface can be configured as a USB connection, e.g., such that the transmitting unit is communicatively coupled to the electronic interface.

For example, the device can be configured to be portable, independently operable, and wirelessly communicative to a remote computer system (e.g., such as a laptop computer, desktop computer, mobile computing device including a smartphone, tablet, or wearable device, and/or a server (e.g., which may be in the cloud). In such examples, the device can be operable to detect the electrophysiological signals (as well as behavioral signals) and process the data from the user wearing the device in a plurality of unrestrictive environments, e.g., including when the user is stationary and when the user is moving. Also, for example, the electrophysiological signals can be evoked from the user by an environmental stimulus in the user's environment. For example, in the exemplary portable, independently operable, and wirelessly communicative device, the data acquisition unit can be detachably coupled to the contact side of the casing unit.

In such examples, the data acquisition unit can be configured to include a disposable electrode sensor assembly including a flexible and adhesive substrate, one or more electrodes attached to the substrate, and an electrical cable that electrically couples the one or more sensors to the signal processing circuit of the data processing unit, e.g., in which the disposable electrode sensor assembly is securably and detachably coupled to the casing unit, and the electrical cable protruding from the disposable electrode sensor assembly. Also, in such examples, the device can include a connection port to electrically couple the electrical cable of the disposable electrode sensor assembly to the signal processing circuit of the data processing unit, in which the connection port includes a retractable cover attached to the casing unit to expose and cover the connection port.

For example, in the exemplary portable, independently operable, and wirelessly communicative device, the data acquisition unit is non-detachably coupled to the contact side of the casing unit. In such examples, the data acquisition unit can be configured to include a moveable electrode containment assembly configured to protrude outward and compressibly retract from the casing unit, in which the moveable electrode containment assembly includes one or more electrodes electrically coupled the signal processing circuit of the data processing unit by an electrical conduit. In some examples, the detected electrophysiological signals are electromyography (EMG) signals sensed from head muscles of the user associated with the user's eye blinking or facial expressions. In some implementations, for example, the determined quantitative information set is presented on a user device including at least one of a laptop or desktop computer, smartphone, tablet, or wearable device by a graphical user interface of an application that interacts with or operates the device.

The exemplary device shown in FIG. 2B includes a data processing unit communicatively coupled to a data acquisition unit configured to contact a user's forehead. The data processing unit is encased in a casing structure 204 or housing, and the data acquisition unit is at least partially encased in the casing. In some embodiments, the data acquisition unit is configured to move with respect to the casing, e.g., when a user makes contact with the data acquisition unit to provide suitable contact of the user's forehead with the sensors of the data acquisition unit.

In one embodiment of the sensory, motor and/or cognitive analysis device shown in FIG. 2B, the device includes a housing configured to include a section that may receive contact from a forehead of a user; a data acquisition unit located in the section of the housing and configured to include one or more sensors to detect electrophysiological signals of a user when the user makes the contact with the device; and a data processing unit located in the housing and in electrical communication with the data acquisition unit, the data processing unit configured to include a signal processing circuit to amplify the detected electrophysiological signals as data and a transmitter to transmit to the data to a remote computer system, in which the device may acquire brain signal data from the user, and wherein a quantitative information set associated with a cognitive or sensory function may be based on the signal data.

Implementations of the exemplary device can include one or more of the following exemplary features. For example, the signal processing circuit of the data processing unit can include an analog-to-digital unit to digitize the amplified signal. For example, the data processing unit can further include a processor to process the data, and a memory to store the data. For example, the device can further include a power supply unit encased within the casing unit and electrically coupled to the data processing unit to provide electrical power. For example, the device can further include an eye-tracking unit including an optical sensor to receive data corresponding to eye blinking of the user. For example, the device can further include a display screen located at a fixed position away from user when in contact with the section of the housing to assist in an eye-tracking application of the eye-tracking unit. For example, the determined quantitative information set can be presented on a user device including at least one of a laptop computer, desktop computer, smartphone, tablet, and/or wearable device by a graphical user interface of an application that interacts with or operates the device.

Examples of an exemplary frontal electrode configuration using minimal electrode sensors to detect desired electrophysiological data are described in PCT Publication WO 2014/059431 A2, entitled “CONFIGURATION AND SPATIAL PLACEMENT OF FRONTAL ELECTRODE SENSORS TO DETECT PHYSIOLOGICAL SIGNALS”, incorporated herein by reference in its entirety, and may be implemented using the disclosed technology. In some examples, the sensor configuration can include a recording electrode, a ground electrode, and a reference electrode, linearly arranged about the user's forehead. The recording electrode can be configured at a first location about the forehead to acquire an electrophysiological signal of the user, the reference electrode is configured at a second location about the forehead to acquire a second electrophysiological signal of the user as a reference signal to the electrophysiological signal, and the ground electrode is configured at a third location about the forehead to acquire a third electrophysiological signal of the user as an electrical ground signal. For example, the arrangements of the three electrodes can be aligned in a substantially straight line along the sagittal direction of the frontal region of the user's head, with the recording electrode (e.g., at the first position) posteriorly positioned to the ground electrode, which is posteriorly positioned to the reference electrode.

In some implementations, for example, the electrophysiological signals detected by the exemplary device can be electroencephalography (EEG) signals sensed from the brain of the user. For example, the EEG signals can be associated with an event-related potential, e.g., based on a stimulus presented to the user wearing the device on the frontal region of the user's head, or by environmental stimuli in the user's environment. In some implementations, for example, the electrophysiological signals detected by the exemplary device can be electromyography (EMG) signals sensed from skeletal muscles (e.g., including facial muscles) of the user. For example, the EMG signals may be resultant from eye blinks of the user, where eye blinks may be in response to an event-related potential based on stimuli presented by a display screen to the user, or by environmental stimuli in the user's environment.

In some embodiments, for example, the data acquisition unit of the exemplary device can include a plurality of recording electrodes configured about the user's forehead or other regions of the user's head to acquire multiple channels of electrophysiological signals of the user. In one example, two (or more) additional recording electrodes may be arranged linearly with respect to the first recording electrode, ground electrode, and reference electrode arranged in the sagittal direction. In another example, one (or more) additional electrodes can be positioned to the left of the first recording electrode, while other additional recording electrode(s) can be positioned to the right of the first recording electrode.

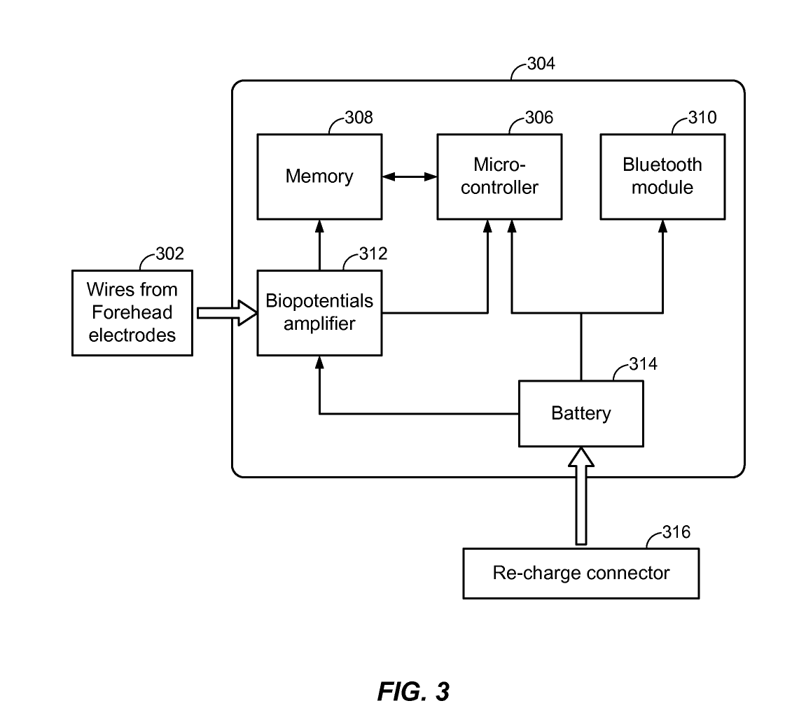

FIG. 3 shows a block diagram of an exemplary data processing unit of the disclosed devices and systems. In the example shown in FIG. 3, the data processing unit 304 includes a processor 306 (e.g., such as a microcontroller or programmable processor) to process data acquired from a user. The processor is in communication with a memory 308 to store the data, a data communications unit 310 (e.g. a Bluetooth module) to transmit and/or receive data, and a signal processing circuit 312 (e.g. a biopotentials amplifier) to amplify, digitize, and/or condition the acquired physiological data obtained from the user. The data may be received from forehead electrodes 302. In one configuration, the data communications unit includes a wireless transmitter/receiver (Tx/Rx) device, e.g., such as a Bluetooth module (not shown). The data processing unit includes a battery 314 (e.g., a power supply) to supply power to the units of the data processing unit. The power supply may be connected to a re-charge connector 316. The elements shown in FIG. 3 may also be defined outside of the data processing unit 304.

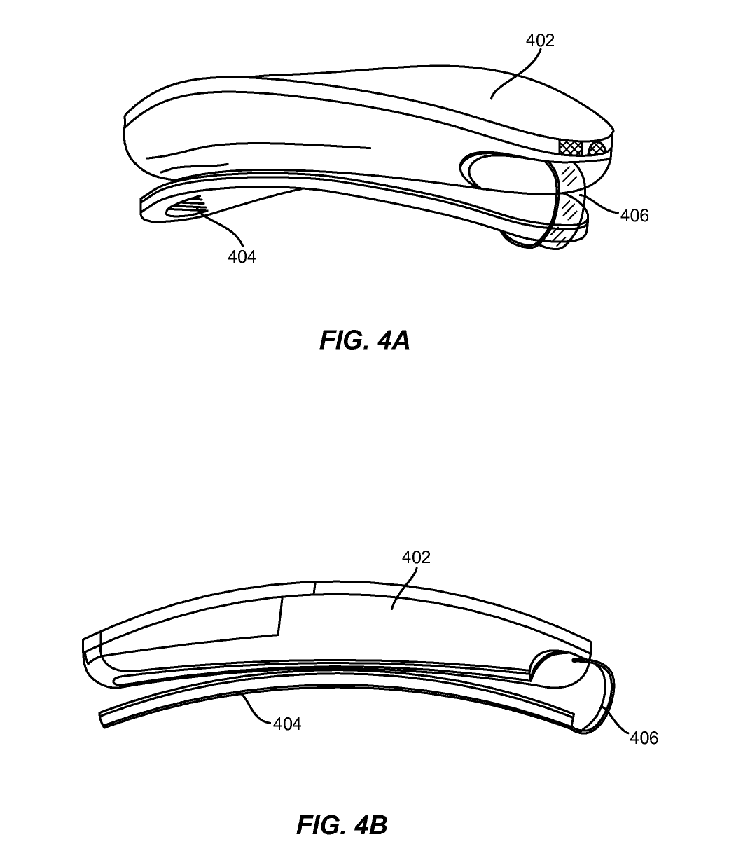

FIGS. 4A, 4B, 5, 6, 7, and 8 show schematic diagrams of an exemplary embodiment of a portable device of the disclosed technology featuring a casing of a data processing unit 402, a detachable data acquisition unit 404 from an encased data processing unit, and an electric connection between the data acquisition and data processing units 406. FIGS. 4A and 4B show three-dimensional schematic diagrams of an exemplary portable device of the disclosed technology featuring a detachable data acquisition unit from an encased data processing unit. As show in this figure, the exemplary unit comprises two parts: a rigid or semi-rigid casing 402 that includes the electronic components and a detachable curved flexible component (a conformable base) 404. This novel device may provide an ergonomic fit for a user's forehead. The materials and design of this unit may improve signal acquisition reliability (e.g. associated with sensor contact quality (signal to noise ratio), and user comfort (light weight, adequate forehead fit, etc.).

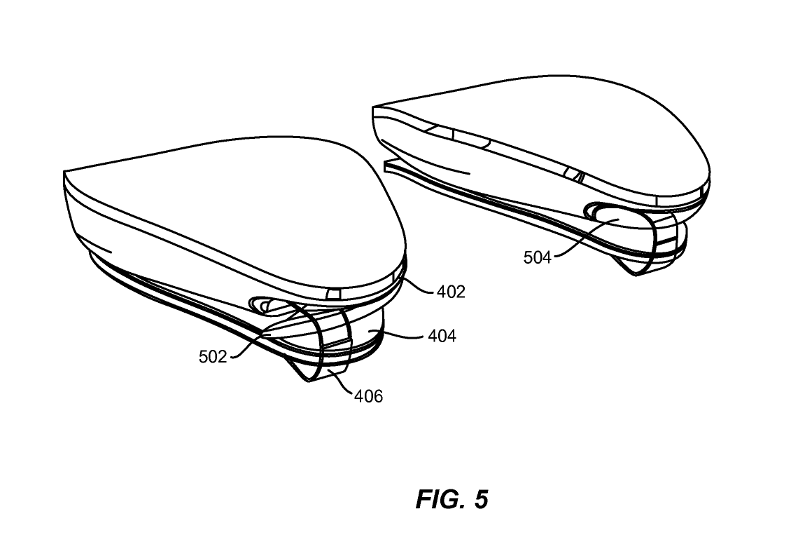

FIG. 5 shows three-dimensional schematic diagrams of the exemplary portable device of FIGS. 4A and 4B showing a retractable cover of the casing structure of the data processing unit in an open 502 and closed 504 configuration that may receive an electrical connection tether to the detachable data acquisition unit. In this exemplary unit the sensor connection was placed to the side, out of the user's FOV (field of view) and protected by a retractable cover, designed so that the connection may be both minimally obtrusive, as well as easy to access, secure, stable and maintaining the aesthetics of the unit. Other possible embodiments include, but are not limited to, the connection between sensors and the data processing unit being hidden from view, defined between the sensors and the flexible component of the unit, or the sensors being included in the processing unit as a semi-permanent fixture establishing contact through the conformable base.

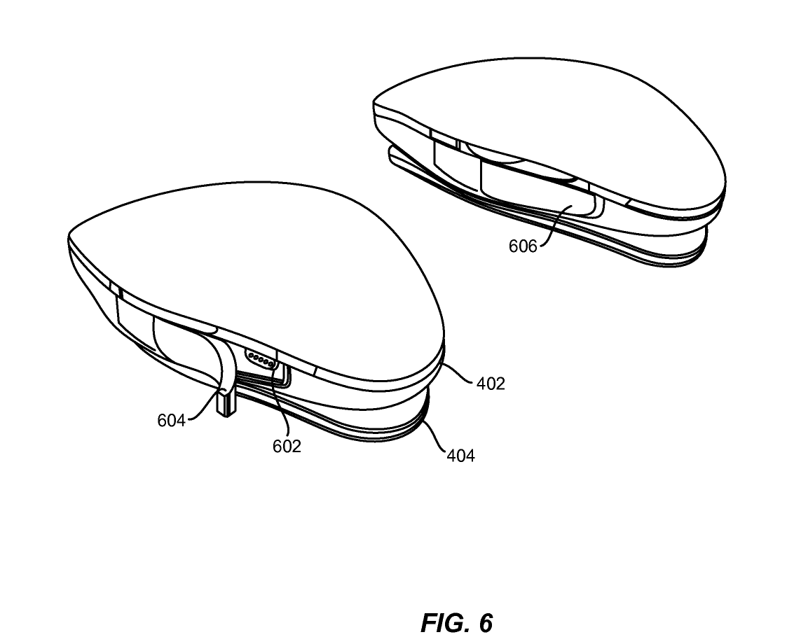

FIG. 6 shows three-dimensional schematic diagrams of the exemplary portable device of FIGS. 4A and 4B showing a retractable cover of the casing structure of the data processing unit in the open 604 and closed 606 configuration that may receive a connector for power and/or data transfer via a power interface 602.

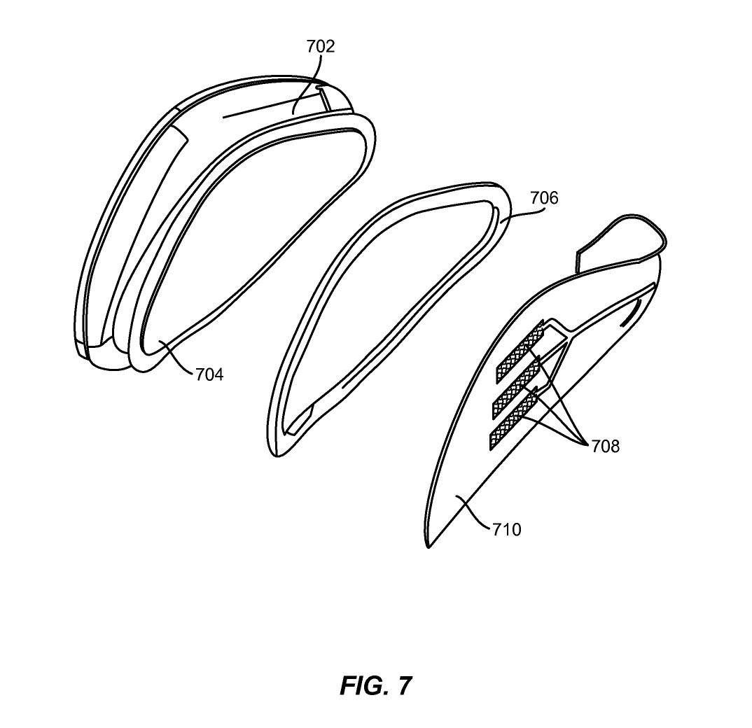

FIG. 7 shows an exploded three-dimensional schematic diagram of an exemplary data processing unit of the exemplary portable device of FIGS. 4A and 4B. This figures shows the previously described exemplary parts (e.g. the base of the processing unit casing 702 and the conformable base 704 of the detachable data acquisition unit), as well as a removable adhesive securement component 706 that may benefit the placement and securing of the device to the user's forehead, maintaining the necessary stability and pressure conducent to adequate signal quality during signal acquisition, while also, by virtue of the design of this embodiment, increasing user comfort as it avoids the traditional headband securement design that may lead to discomfort and headaches.

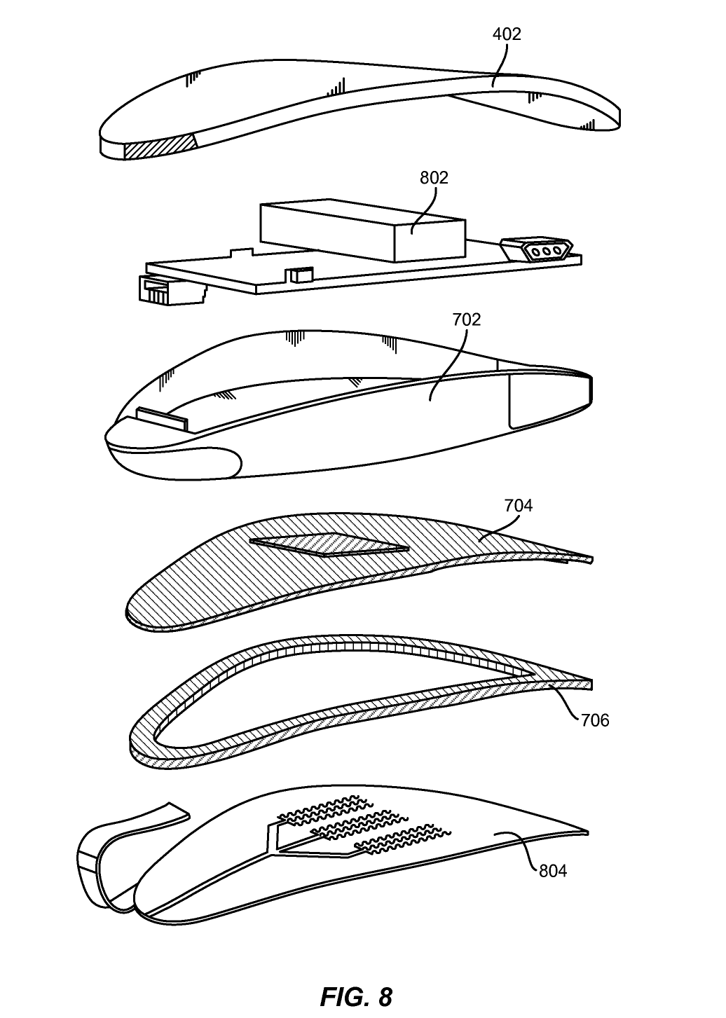

FIG. 8 shows an exploded three-dimensional schematic diagram of an exemplary data processing unit and data acquisition unit of the exemplary portable device of FIGS. 4A and 4B, including the casing cap of the data processing unit 402, electronic components of the data processing unit 802, the casing base of the data processing unit 702, the detachable conformable base of the data acquisition unit 704, the securement component 708 (e.g. removable adhesive band), and a detachable and disposable conformable sensor assembly of the data acquisition unit 804.

One particular advantage of the disclosed exemplary embodiment is conferred by the adhesive securement to the user's forehead. This obviates the need for structural support beyond the forehead (e.g. straps or bands) for application and/or usage of the exemplary device. In the disclosed embodiment, the conformable sensor assembly is adhesively secured to the forehead. A double-sided adhesive secures the sensor assembly to the conformable base of data acquisition unit. Because the exemplary device is lightweight, the sensory assembly adhesive is sufficient to maintain the position of the sensor assembly on the forehead during use. Furthermore, the double-sided adhesive is sufficient to reliably and securely maintain contact between the sensor assembly and the data acquisition unit. These aspects of the disclosed exemplary embodiment may provide improved signal quality and user comfort in comparison to previous designs. The user comfort further benefits from the conformable base of the disclosed exemplary embodiment.

Prior designs may adhesively secure the sensor assembly to the user's forehead, but not in combination with an encased lightweight data processing unit or conformable base. The functionality of such a system may then depend on additional devices. Alternatively, prior designs may combine data processing and a sensor assembly in an integrated unit, but not in a lightweight manner that can be secured in place with an adhesive as can the disclosed embodiment. Such systems may rely on straps or bands that may cause headaches or other discomforts for the user.

In an alternative embodiment of the disclosed technology, the sensor assembly may be incorporated into the data acquisition unit. In this alternative embodiment, the double-sided adhesive may be used to reliably and securely maintain contact between the data acquisition unit and the forehead.

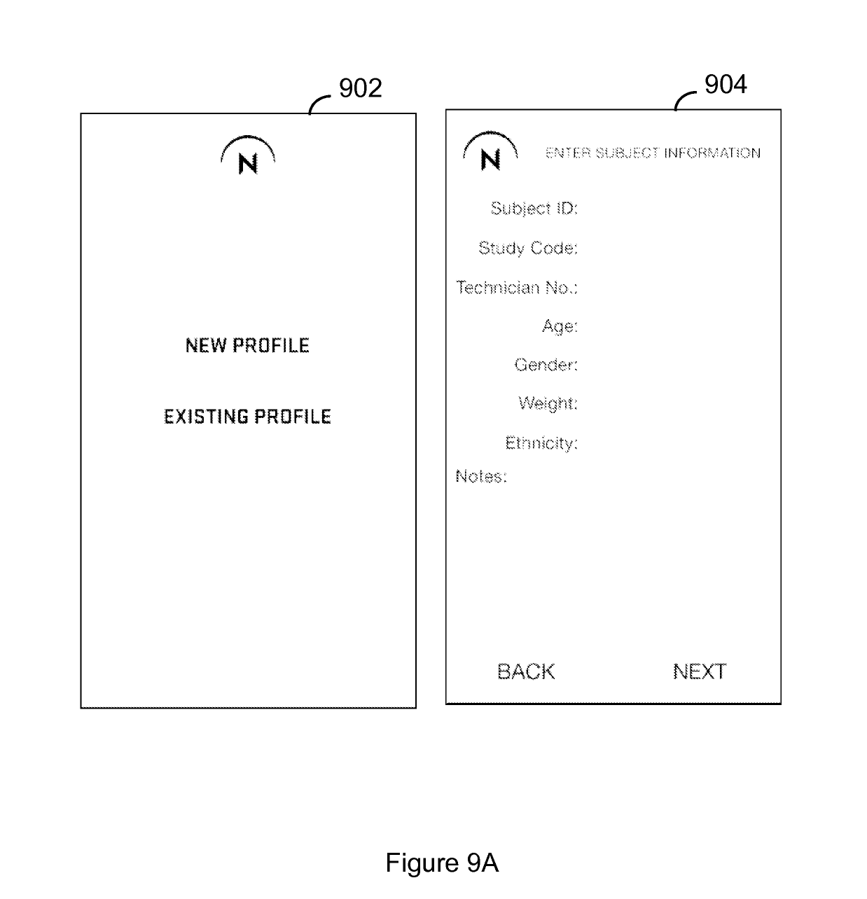

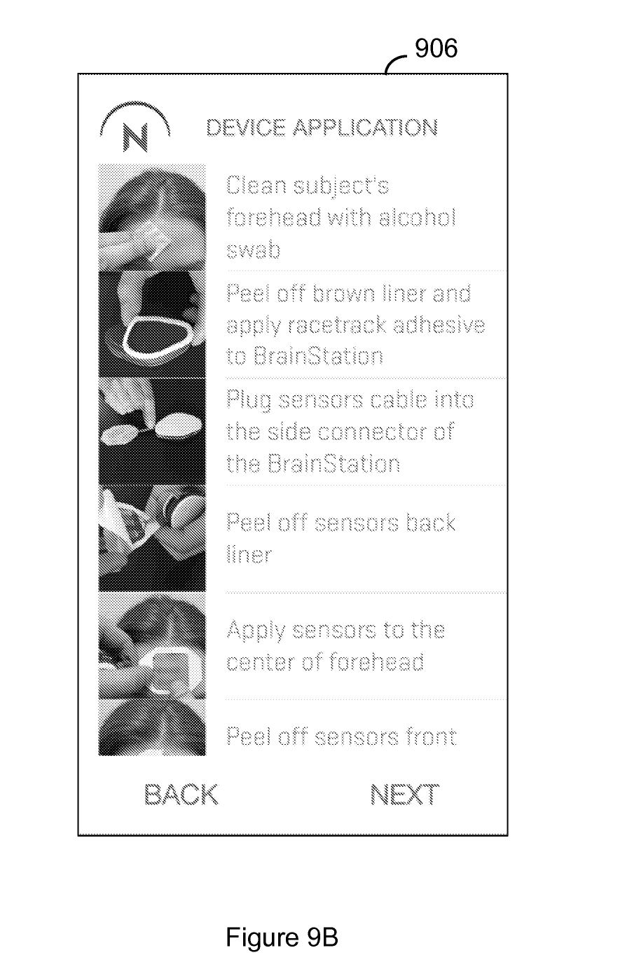

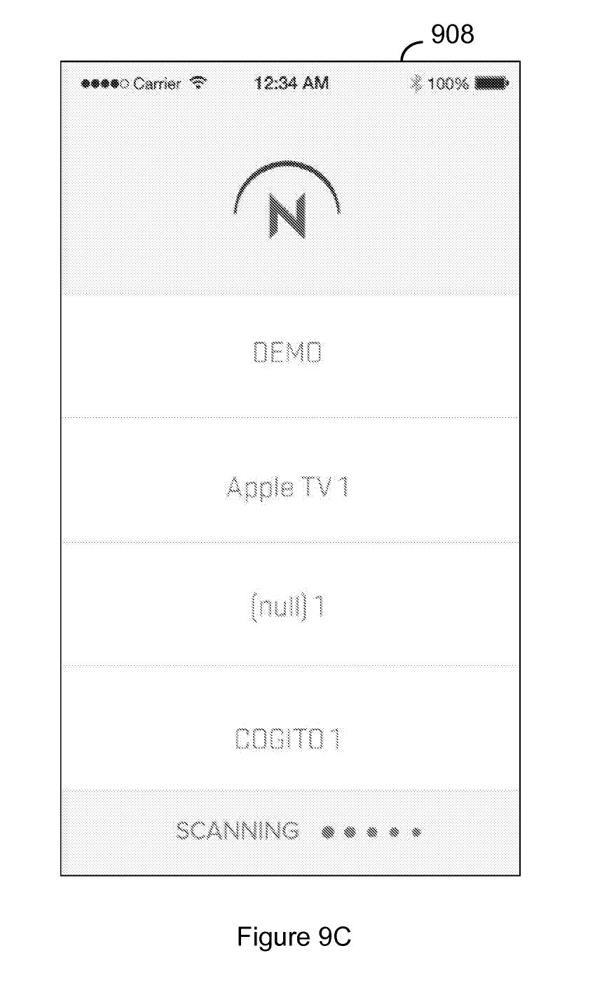

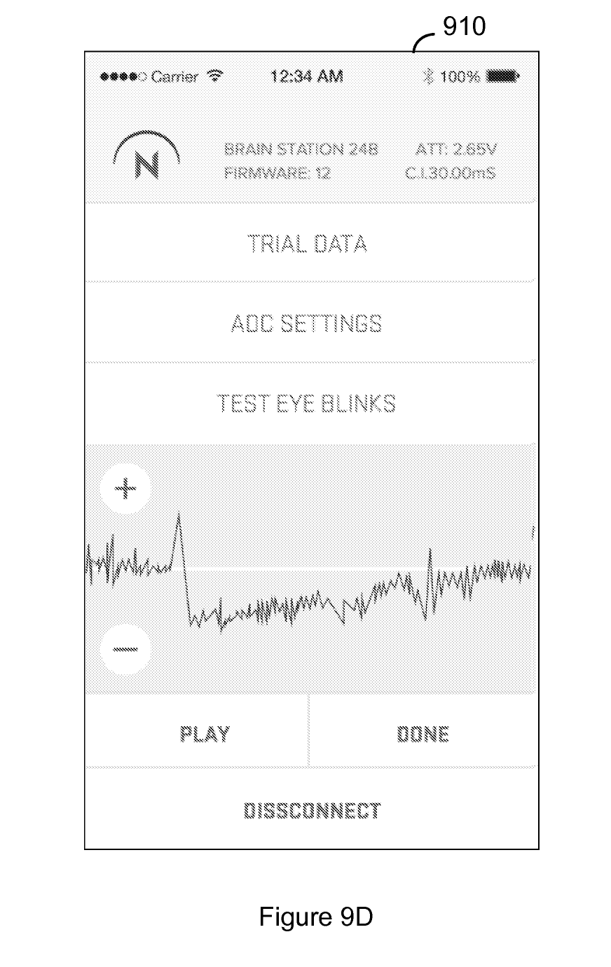

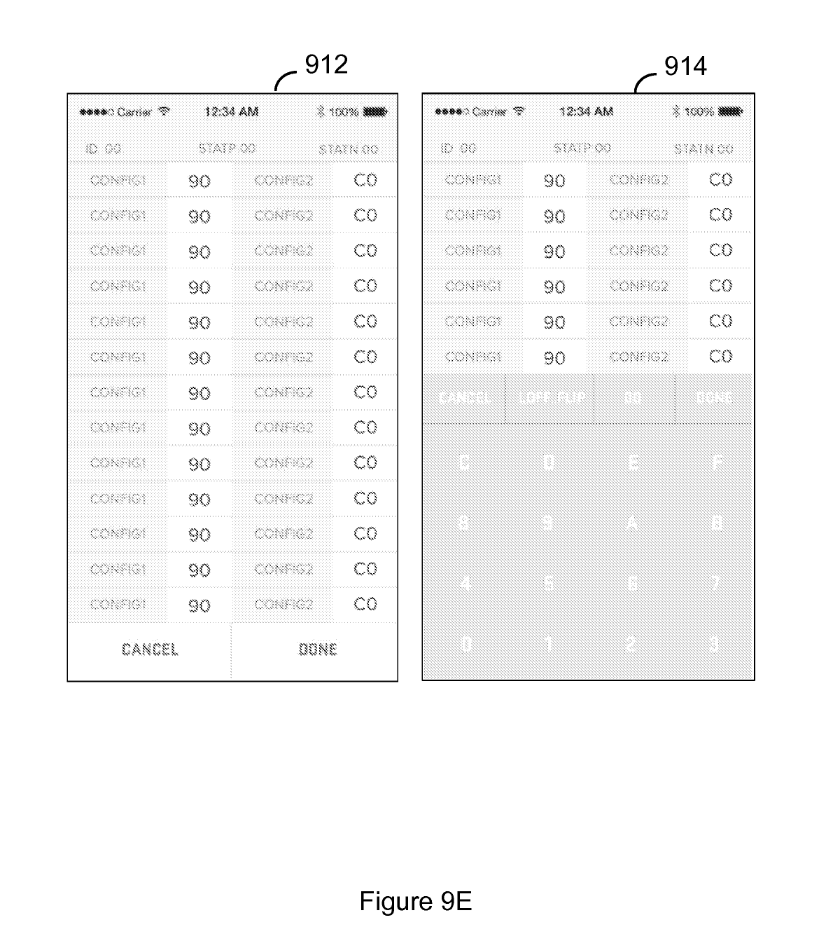

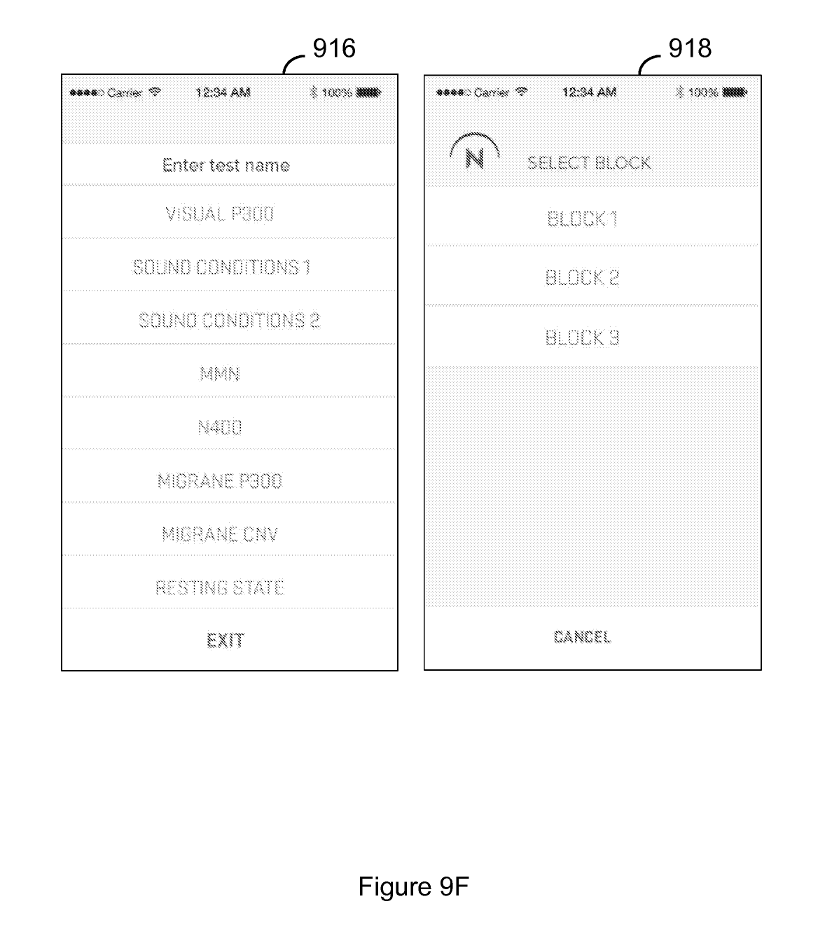

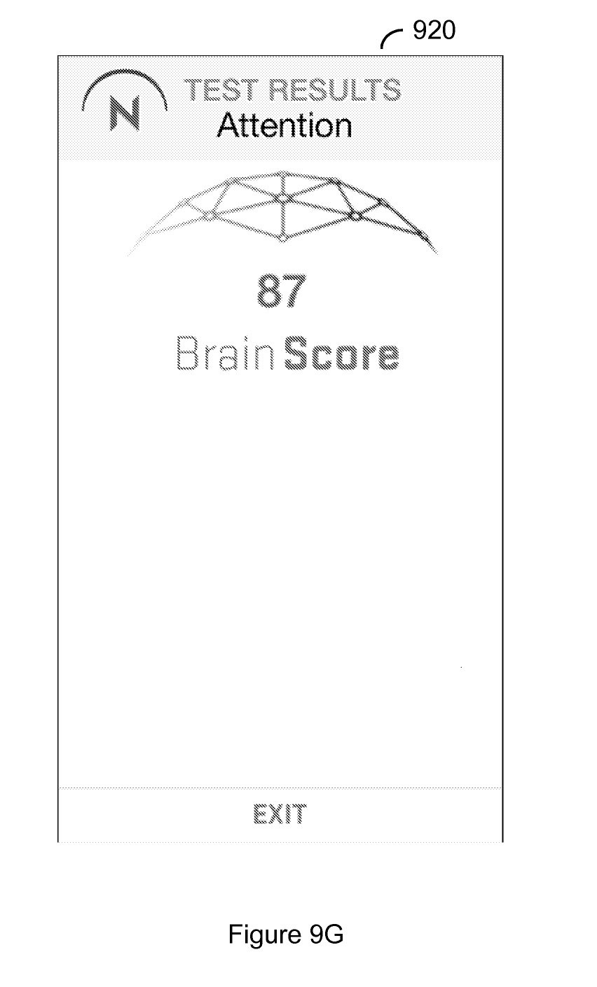

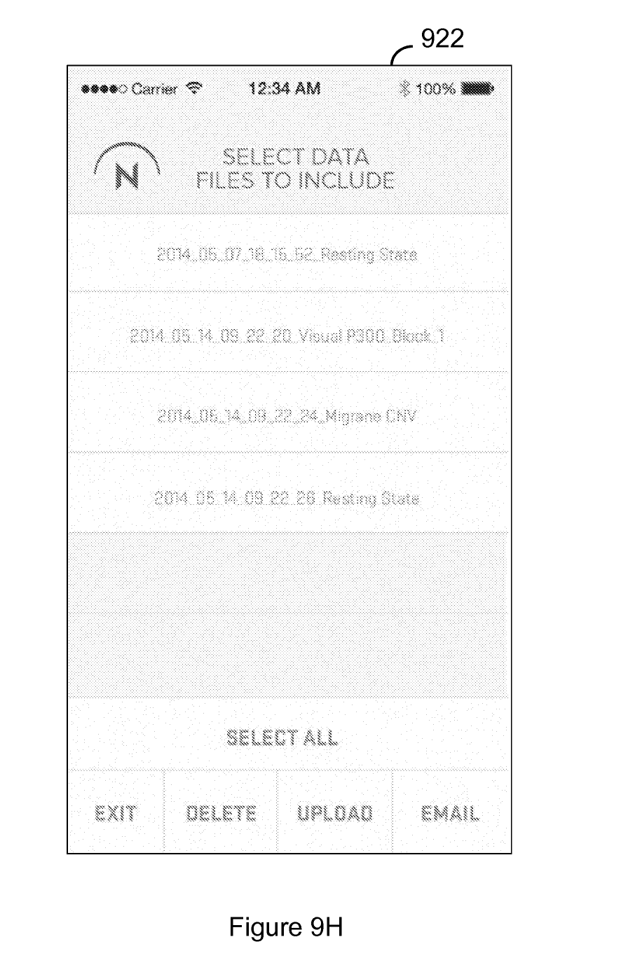

FIG. 9A shows an illustration of a display screen 902 of an exemplary user interface of a software application of the disclosed technology in which a user can either create a new user profile or use an existing profile. The user profile 904 can be used to associate acquired data with user-specific information (such as but not limited to name or identification number) as well as demographic information (such as but not limited to age, gender, weight, and/or ethnicity). This enables an easy-to-use detailed user profile that can be associated with repetitive testing. FIG. 9B shows an illustration 906 of a sliding display screen of an exemplary user interface of a software application of the disclosed technology in which device application instructions of the disclosed, exemplary device are shown. These instructions can be used by either lay or professional/specialized users to apply the device to the user's forehead. For example, the structural design of the exemplary device shown in FIGS. 4A-8 allows a lay user to apply the device to his/her forehead and operate the device. The exemplary interface 906 depicts example instructions for the user to align and attach the device to his/her forehead to enable data acquisition, e.g., without the need for a professional or specialized technician to apply the device. The interface, paired with the described device and multiple tests, allows for a “brain assessment” tool that can be used outside of specialized environments without the need of a professional team. Rather, the tool and/or device may be used by users in any environment, such as home, schools, and/or coffee shops. FIG. 9C shows an example of a display screen 908 of an exemplary user interface of a software application of the disclosed technology in which a user can wirelessly select and connect the software application to a specific portable device. As shown in FIG. 9C, the user may connect to software applications or devices such as an APPLE TV. Other devices may include medical devices or gaming units. FIG. 9D shows an example of a display screen 910 of an exemplary user interface of a software application in which a user can view the acquired and processed EEG signal as shown by a visual representation of voltage as a function of time. The real time display of the EEG system allows for an improved visual evaluation of the EEG signal quality being acquired, so that any adjustments for quality improvement can be timely made, if necessary. As shown in FIG. 9D, the user may select various settings and menus, such as trial data, ADC settings, and test eye blinks. In some embodiments, the display screen can also serve as a home navigation screen, in which other display screens of the user interface may be accessed and viewed, and/or the software application can be disconnected from the portable device. FIG. 9E shows illustrations of display screens 912 and 914 of an exemplary user interface of a software application of the disclosed technology in which a user can view and/or edit specific settings related to the operations and/or functionality of the data acquisition and processing units. FIG. 9F shows illustrations of display screens 916 and 918 of an exemplary user interface of a software application of the disclosed technology in which a user can select from a variety of testing assessments. This interface enables a selection from within a growing repertoire of possible neural—mental performance tests, giving the user control over which tests to choose from, as well as maintaining access and visual display of possibilities. The “battery” of tests included in this exemplary embodiment provides in depth assessment of a variety of sensory, motor and/or cognitive processes, altogether contributing to the user's expanded neuro-cognitive profile. In some embodiments, a specific assessment may have multiple sub-assessment options to choose from FIG. 9G shows an illustration of a display screen 920 of an exemplary user interface of a software application of the disclosed technology in which a user can visualize the results of a test with the subsequent neurocognitive profile performance assessment (e.g. BrainScore), providing both real-time and/or delayed feedback on the user's assessment. Quantitative assessments can be made for each testing trial per testing type and/or result from the combination of multiple tests. FIG. 9H shows an illustration of a display screen 922 of an exemplary user interface of a software application of the disclosed technology in which a user can view and select one or more recorded data sets for data transfer/upload, processing, analysis, and/or storage. In some embodiments, the cognitive, motor, and/or sensory profile results may or may not be presented to the user.

Altogether, this exemplary system interface provides a functional and aesthetical design that improves portable and use of the exemplary system to perform sensory, motor, cognitive and/or emotional state assessments of the user outside of specialized environments and at any time without the need for specialized personnel or user's professional knowledge.

Some exemplary features and implementation advantages of the disclosed devices and systems include the following. For example, the disclosed devices and systems can be operated by general users outside of a clinical setting, with safety and accuracy. This capability, which is enabled by the design and functionality of the disclosed devices and systems, allows the user freedom-of-use in a wide variety of contexts and locations, giving rise to an expanded pool of users and subjects and new types of physiological and/or behavior response analyses paradigms. Additionally, such freedom-of-use of the disclosed devices and systems can reduce the cost and specifications of use for brain monitoring systems. For example, the disclosed portable devices and methods can be used by non-experts to place the exemplary portable device on the forehead of evaluated persons (or allow the subjects to place the exemplary portable device on themselves) to acquire physiological and/or behavioral signals (e.g., which can be associated with event-related potentials in some implementations, or the user's environmental context in other implementations), and thereby provide a unique cognitive, motor and/or sensory profile of the subject or subjects. For example, the disclosed devices and systems enables operation by such non-experts, no longer specifying that the operator to be neuroscientists, psychologists, or specialized physicians to implement the physiological and/or behavioral data acquisition or to interpret the generated cognitive and/or sensory profile information of the user that is provided to the user in the analysis of the acquired physiological and/or behavioral data. For example, the non-expert users can implement the disclosed devices and systems to obtain awareness and mental information profiles of the evaluated person(s), e.g., either themselves or others. For example, application and operation of such devices and systems can be performed by the user following instructions, without a need for technical expertise to apply or operate the device or system. Additionally, for example, implementations of the disclosed devices, systems and methods can also be used within the context of brain-machine interfaces and thereby expand the possible applications of such systems.

Implementations of the subject matter and the functional operations described in this disclosure may be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer include one or more processors for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed:

1. A sensory, motor and/or cognitive analysis device, comprising:
a portable casing unit having aa contact side conformable to a forehead region of a user;
a data acquisition unit including one or more sensors configured to detect electrophysiological signals of the user when the user makes contact with the device;
a data processing unit within the casing unit and in communication with the data acquisition unit, the data processing unit including a signal processing circuit to amplify and digitize detected electrophysiological signals as data, a processor to process the data, a memory to store the data, and a transmitter to transmit the data to a remote computer system; and
a power supply unit within the casing unit and electrically coupled to the data processing unit to provide electrical power,
wherein the device is operable to acquire physiological and/or behavioral signal data from the user; and wherein a quantitative and/or qualitative information set associated with a cognitive or sensory assessment is based on the signal data.

2. The device of claim 1, wherein the detected electrophysiological signals are electroencephalography (EEG) signals measured from a brain of the user.

3. The device of claim 2, wherein the data processing unit is configured to determine the quantitative information set based on the measured EEG signals associated with an event-related potential (ERP).

4. The device of claim 1, wherein the power supply unit includes a rechargeable battery, and the device further comprises an interface assembly including an electronic interface electrically coupled to the battery and a retractable cover attached to the casing unit to expose and cover the electronic interface.

5. The device of claim 4, wherein the electronic interface comprises a USB connection, and wherein the transmitting unit is communicatively coupled to the electronic interface.

6. The device of claim 1, wherein the device is portable, independently operable, and configured to wirelessly communicate to the remote computer system.

7. The device of claim 6, wherein the device is operable to detect the electrophysiological and/or behavioral signals and process the data from the user wearing the device in a plurality of unrestrictive environments including when the user is stationary and when the user is moving.

8. The device of claim 7, wherein the electrophysiological and/or behavioral signals are evoked from the user by an environmental stimulus in the user's environment.

9. The device of claim 6, wherein the data acquisition unit is detachably coupled to the contact side of the casing unit.

10. The device of claim 9, wherein the data acquisition unit is structured to include a disposable electrode sensor assembly including a flexible and adhesive substrate, one or more electrodes attached to the substrate, and an electrical cable that electrically couples the one or more sensors to the signal processing circuit of the data processing unit, the disposable electrode sensor assembly being securably and detachably coupled to the casing unit, and the electrical cable protruding from the disposable electrode sensor assembly.

11. The device of claim 9, wherein the device includes a connection port to electrically couple the electrical cable of the disposable electrode sensor assembly to the signal processing circuit of the data processing unit, wherein the connection port includes a retractable cover attached to the casing unit to expose and cover the connection port.

12. The device of claim 6, wherein the data acquisition unit is non-detachably coupled to the contact side of the casing unit.

13. The device of claim 12, wherein the data acquisition unit is structured to include a moveable electrode containment assembly configured to protrude outward and compressibly retract from the casing unit, the moveable electrode containment assembly including one or more electrodes electrically coupled to the signal processing circuit of the data processing unit by an electrical conduit.

14. The device of claim 1, wherein the detected electrophysiological signals are electromyography (EMG) signals sensed from head muscles of the user associated with the user's eye blinking or facial expressions.

15. The device of claim 1, wherein the determined quantitative and/or qualitative information set is presented on a user device including at least one of a laptop computer, desktop computer, smartphone, tablet, or wearable device by a graphical user interface of an application that interacts with or operates the device.

16. A sensory, motor and/or cognitive analysis device, comprising:
a housing structured to include a section receiving contact from a forehead of a user;
a data acquisition unit located in the section of the housing and structured to include one or more sensors to detect electrophysiological signals of the user when the user makes the contact with the device; and
a data processing unit located in the housing and in electrical communication with the data acquisition unit, the data processing unit structured to include a signal processing circuit to amplify the detected electrophysiological signals as data and a transmitter to transmit the data to a remote computer system,
wherein the device is operable to acquire brain signal data from the user, and wherein a quantitative and/or qualitative information set associated with a cognitive or sensory assessment is based on the signal data.

17. The device of claim 16, wherein the data processing unit further includes a processor to process the data, and a memory to store the data.

18. The device of claim 17, further comprising:
a power supply unit encased within the casing unit and electrically coupled to the data processing unit to provide electrical power.

19. The device of claim 16, further comprising:
an eye-tracking unit including an optical sensor to receive data corresponding to eye blinking of the user.

20. The device of claim 19, further comprising:
a display screen located at a fixed position away from user when in contact with the section of the housing to assist in an eye-tracking application of the eye-tracking unit.

21. The device of claim 16, wherein the determined quantitative information set is presented on a user device including at least one of a laptop computer, desktop computer, smartphone, tablet, or wearable device by a graphical user interface of an application that interacts with or operates the device.

22. The device of claims 16, wherein the user is a human or a non-human animal.
